(19) United States
(12) Patent Application Publication
(10) Pub. No.: US 2010/0185106 A1
(43) Pub. Date: Jul. 22, 2010

(54) LIGHT-EMITTING APPARATUS, PARTICULARLY FOR FLOW MEASUREMENTS

(75) Inventors: Jan Frederik Suijver, Eindhoven (NL); Mischa Megens, Eindhoven (NL); Drazenko Babic, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/376,630

(22) PCT Filed: Aug. 6, 2007
(86) PCT No.: PCT/IB07/53082

§ 371 Date: Feb. 6, 2009

(30) Foreign Application Priority Data
Aug. 9, 2006 (EP) 06118675.5

(51) Int. Cl. A61B 5/02

(52) U.S. Cl. 600/504

(57) ABSTRACT

The invention relates to a light-emitting apparatus (100) comprising an optical waveguide, particularly an optical fiber (1), for guiding a primary light beam (Bₚᵣᵢₘ) into a light-splitting unit (101) which splits it into two or more partial light beams (B1, B3, B4) which are emitted in different directions and have different optical qualities, e.g. different spectral compositions or polarizations. The apparatus may optionally comprise a detector (4) for determining a Doppler shift (Δλᵢ) in reflected light reentering the light-splitting unit (101). This renders it possible to measure simultaneously two or more spatially independent vector components of the flow velocity of a fluid, particularly of blood, surrounding the light-splitting unit (101).

The invention relates to a light-emitting apparatus comprising means for emitting a light beam which was conducted by an optical waveguide. Moreover, it relates to a method for measuring a flow velocity of a medium, particularly of blood.

The measurement of blood flow velocity is gaining increasing importance not only in scientific research, but also in everyday clinical applications. Thus the treatment of aneurysms, for example, can be considerably improved if the blood flow around and in the aneurysm can be accurately assessed. A fiber-optical sensor for remote flow measurements is disclosed in WO 97/12210, wherein said sensor comprises a first optical fiber for guiding a light beam to a reflective surface, from which it is directed through a window into the surrounding medium. Backscattered light from the medium can then reenter the same window and reach a detector via a second optical fiber, which detector measures a Doppler shift in this light. This renders it possible to calculate the flow velocity of the surrounding medium in the direction of the emitted light.

Taking this situation as a starting point, it is an object of the present invention to provide means for a more versatile examination of fluids by means of emitted light. In particular, it is envisaged to measure independent components of the flow velocity vector simultaneously.

This object is achieved by a light-emitting apparatus according to claim 1 and by a method according to claim 11. Preferred embodiments are disclosed in the dependent claims.

The light-emitting apparatus according to the present invention comprises the following components:
a) An optical waveguide for conducting a primary light beam. The optical waveguide may particularly be realized by an optical fiber, and the primary light beam may originate from any suitable source (including e.g. collected ambient light).
b) A light-splitting unit for splitting a primary light beam conducted by said optical waveguide into at least two partial light beams that have different optical qualities and that are emitted in different directions. The expression “optical quality” in this context denotes some inherent physical property of a light beam such as its polarization or its spectral composition.

The described light-emitting apparatus has the advantage of allowing a compact design by using one optical waveguide for conducting a (primary) light beam. At the same time, the apparatus provides two (partial) light beams emitted in different directions that allow manipulations or investigations in at least two spatially independent dimensions. Moreover, the different optical qualities of said partial light beams provide a means for distinguishing their effects in the surrounding medium. As the spreading of light is generally reversible, it is also possible for light from the surroundings to be taken up by the light-splitting unit and to be directed into the optical waveguide. This effect is exploited in preferred embodiments of the invention; but in general the apparatus may merely be used only for emitting light, not for re-collecting it.

There are various possibilities for building the light-splitting unit. In preferred embodiments of the invention, it may comprise, for example, at least one splitting component that is realized by a dichroic beam splitter, a grating, and/or an optical polarizer, such that the splitting component splits an incident light beam (for example the primary light beam) into a first and a second partial light beam of different directions and different optical qualities. Thus a dichroic beam splitter and a grating will split an incident light beam into two beams of different spectral compositions, while the polarizer will split an incident light beam into two beams of different polarizations.

If in the cases mentioned above there is only one splitting component in the light splitting unit which will typically generate only two emitted partial light beams from the primary light beam. In order to emit more partial light beams, the light-splitting unit may comprise a further splitting component (such as a dichroic beam splitter, a grating, and/or an optical polarizer) for splitting the second partial light beam that was generated by the (first) splitting component into a third and a fourth partial light beam of different directions and optical qualities. It should be noted in this respect that the choice of the second partial light beam as an input for the second splitting component does not restrict the design of the apparatus, as the numbering of the first and the second partial light beam leaving the first splitting component is arbitrary. The first, third, and fourth partial light beam are preferably oriented in different directions that do not lie in a common plane, i.e. they issue from the light-emitting apparatus in three spatially independent dimensions.

If two dichroic beam splitters are arranged in series as described above, they may preferably have the shape of a prism with a triangular base and be oriented at a rotational angle of approximately 45° about the axis of an incident beam. In this case the first, third, and fourth partial light beams leaving the light splitting unit will substantially be directed in three mutually orthogonal directions.

If the light splitting unit comprises a grating, this has preferably a blaze angle for a particular wavelength. In this case the light of the incident light beam having said particular wavelength will be refracted by the grating in a certain direction, whereas the residual light of the incident light beam will pass the grating substantially unaffected.

In another embodiment of the invention, the light-emitting apparatus comprises a detector for detecting a secondary light beam that comprises light which was taken up by the light-splitting unit from its surroundings. In this case, the light-emitting apparatus may be used not only for emitting light into a medium, but also for sensing and evaluating light coming from said medium.

In a further development of the above embodiment, the detector is adapted to process components of the secondary light beam of different optical qualities separately. Said components are therefore treated independently, which preserves any information carried by these components. A particularly important application of this design is found in the case in which the components of the secondary light beam originate from the different partial light beams leaving the light-splitting unit. It is then possible, for example, to observe the effects of the partial light beams independently.

In another version of the light-emitting apparatus with a detector, said detector comprises an evaluation module for determining a Doppler shift in at least one component of the secondary light beam with respect to a corresponding partial light beam. Measuring the Doppler shift that a partial light beam undergoes when it is reflected by e.g. a particle in the surrounding medium renders it possible to determine the velocity of said particle in the direction of the partial light beam. If the detector is adapted to determine the Doppler shifts of all light components of the secondary light beam that originate from different partial light beams, it is therefore possible to measure as many spatially independent components of the flow velocity of the surrounding medium as there are partial light beams. The use of three partial light beams will thus offer a complete determination of the three-dimensional flow velocity vector.

The light-emitting apparatus may further comprise a light source for emitting the primary light beam into the optical waveguide, which emitted primary light beam should be composed of light having various optical qualities which can be separated into the partial light beams by the light-splitting unit. The light source may particularly be a laser.

If the light source is a laser, it should have a coherence length greater than 1 mm, preferably greater than 10 mm, most preferably greater than 100 mm. In this case the primary light beam generated by light will be suitable for Doppler measurements.

In a particular embodiment of the light-emitting apparatus, the light-emitting apparatus is developed as a medical device, particularly a catheter device or an endoscope device, for use in a medical diagnosis or treatment procedure which may be a non-invasive, minimally invasive (e.g. endoscope-based), or invasive (surgical) procedure. The catheter device or endoscope device may solely consist of the light-emitting apparatus, or the light-emitting apparatus may be incorporated into a catheter device or endoscope device that comprises additional features known to those skilled in the art.

The invention further relates to a method of measuring a flow velocity of a fluid, particularly of blood, comprising the following steps:
a) Emitting at least two partial light beams of different optical qualities from a measuring location (inside the fluid) in different directions.
b) Receiving a secondary light beam that comprises components consisting of light from the partial light beams which were reflected in the fluid.
c) Determining a flow velocity of the fluid (or at least of those constituents of the fluid that reflected a partial light beam) from a Doppler shift in said components of the secondary light beam.

The method in a general form comprises the steps that can be executed with a light-emitting apparatus of the kind described above. Therefore, reference is made to the preceding description for more information on the details of, advantages of, and improvements offered by this method.

These and other aspects of the invention will be apparent from and elucidated with reference to the embodiment(s) described hereinafter. These embodiments will be described by way of example with the help of the accompanying drawings, in which:

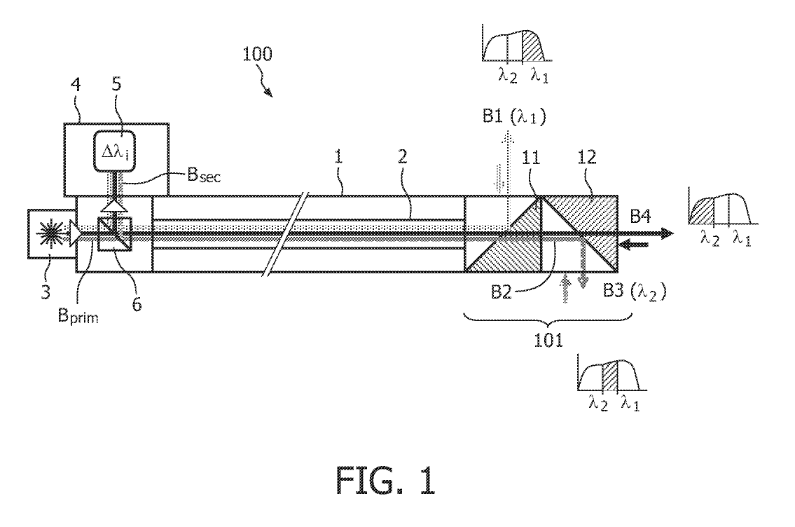

FIG. 1 schematically shows a light-emitting apparatus for blood flow measurements according to a first embodiment of the invention, comprising two dichroic beam splitters;

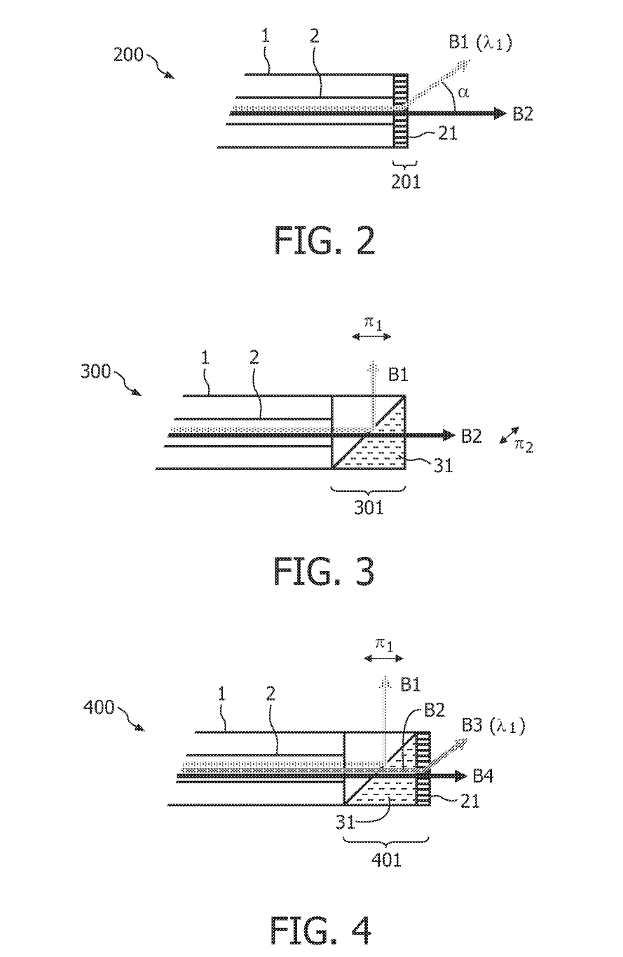

FIG. 2 schematically shows a light-emitting apparatus according to a second embodiment of the invention, comprising a grating;

FIG. 3 schematically shows a light-emitting apparatus according to a third embodiment of the invention, comprising an optical polarizer; and

FIG. 4 schematically shows a light-emitting apparatus according to a fourth embodiment of the invention, comprising an optical polarizer and a grating arranged in series.

Like reference numbers in the Figures and reference numbers that differ by integer multiples of 100 refer to identical or similar components.

Accurate and reliable measurements of blood flow are required in large number of clinical settings, for example:
The definition of the blood flow obstruction severity in atherosclerotic stenotic disease in cranial vessels, head and neck vessels, thoracic and abdominal vessels, and vessels of the lower limb. Determination of blood flow alterations prior to and after the endovascular or surgical treatment is of a particular importance.
Techniques for functional assessment of blood flow dynamics in individual micro vessels, which techniques are likely to become tools of increasing importance, for example in the evaluation of new vasoactive drugs.
Measurement of the blood flow in and around intracranial aneurysms and AVMs prior to and after endovascular or surgical treatments, which would provide answers as to the applicability of a used approach as well as define blood flow alterations in terms of blood velocity and wall shear stress prior to and after the treatment.
Detection of blood flow changes in malignant and benign tumors as an indicator of tumor growth (e.g. the localization of blood vessels within an ovarian tumor and the presence or absence of a diastolic notch are the most useful variables in the evaluation of ovarian tumors).

Assessment of the blood flow as a predictor of aneurysm formation and growth as well as the dynamic assessment of the blood flow inside an aneurysm pouch is crucial in order to understand and predict aneurysm behavior. Well-understood and clinically proven and reproducible flow assessment would potentially improve the ability of a vascular interventionalist or vascular interventional physician to define the optimum treatment strategy. The fundamental question here is to determine whether or not intervention in a particular patient is required. This translates into the problem of determining the blood flow velocity inside the artery as well as in the aneurysm. A comparison of these values, as well as their fluctuations over time, renders it possible to assess the risk associated with a particular aneurysm.

There are several techniques that can be used to assess the intracranial blood flow and intra-aneurysmal flow pattern, for example color flow US, CT imaging, MR imaging,

SPECT imaging, and PET imaging. None of these techniques, however, meets the clinical requirements regarding accuracy, simplicity, cost-effectiveness, resolution, and robustness. In the following, therefore, various embodiments of a light-emitting apparatus according to the present invention will be described that are particularly adapted for blood flow measurements. The apparatuses allow real-time blood flow read-out performed with an endovascular optical fiber sensor located proximally to the targeted anatomy or in the anatomy itself. More specifically, the apparatuses comprise a single fiber in a catheter in combination with specially constructed optical elements to enable a three-dimensional flow velocity measurement in its vicinity. The new velocimetry technology renders a detection and display of the blood flow speed in various directions in the vicinity of the probe possible.

FIG. 1 is a schematic representation of a first embodiment of a light-emitting apparatus in the form of a catheter device 100 for blood flow measurements, wherein only the components important for the present invention are shown. The catheter device 100 comprises a single-mode core waveguide 1 consisting of a fiber core 2 embedded in a fiber cladding. At a first end of the waveguide 1 (left side in the Figure), a laser 6 is arranged as a light source, sending a primary light beam Bₚᵣᵢₘ via a beam splitter 6 into the fiber core 2.

At the opposite end of the waveguide 1 (right side in the Figure), a light-splitting unit 101 is arranged that splits the primary light beam Bₚᵣᵢₘ into three partial light beams B1, B3, and B4 which are emitted in three different directions (in the situation shown, these directions will be mutually perpendicular, e.g. beams B1 and B4 lie in the plane of drawing while beam B3 projects vertically from said plane). The splitting is based on the distinct optical qualities of the partial light beams which together constitute the primary beam Bₚᵣᵢₘ. In the embodiment shown, said optical quality is the spectral composition of the light beams, and the light splitting unit 101 consists of two dichroic beam splitters 11 and 12 that have the shape of a prism and that are rotated with respect to each other through an angle of approximately 45° about the axis of the primary beam Bₚᵣᵢₘ. At the first dichroic beam splitter 11, the first partial light beam B1 (comprising the part of the spectrum of the incident beam Bₚᵣᵢₘ with wavelengths≧λ₁) is reflected, while the residual light is transmitted as an intermediate partial light beam B2. At the second dichroic beam splitter 12, the third partial light beam B3 (comprising the part of the spectrum of the incident beam B2 with wavelengths≧λ₂, with λ₂<λ₁) is reflected, while the residual light is transmitted as a fourth partial light beam B4.

The two wavelengths λ₁ and λ₂ above which the respective dichroic elements 11, 12 are reflective may lie relatively close together (closer than about 100 nm), which has the advantage that the optical properties of the blood will be substantially independent of wavelength in this range. Alternatively, the wavelengths may be further apart, facilitating the construction of the dichroic mirrors 11, 12. The choice of wavelength will ultimately depend on the optical properties of the human blood, such as the transmission window and scattering efficiencies. Naturally, there is a design freedom in choosing the wavelengths of the various partial beams by selecting the filter pass bands, i.e. the short wavelength may be reflected first and the long wavelength transmitted to the end face, instead of the situation shown in FIG. 1, where the long wavelength is reflected first and the short wavelength is transmitted to the end face.

Small arrows in FIG. 1 further indicate that light of the partial light beams reflected in the surrounding blood (for example by cells) is taken up by the light-splitting unit 101 and travels as a secondary light beam B_{sec} in opposite direction through the optical fiber 1 to the primary light beam Bₚᵣᵢₘ. The secondary light beam B_{sec} is then directed by the beam splitter 6 into a detector 4, in which an evaluation unit 5 is adapted to determine the Doppler shift Δλᵢ independently for the three components of the secondary light beam B_{sec} that originate from the different emitted partial light beams B1, B3, and B4. The separation of the components of the secondary light beam B_{sec} can be achieved inside the detector 4 by a device similar to the light-splitting unit 101.

The Laser Doppler velocimetry performed by the evaluation unit 5 uses the frequency shift produced by the Doppler effect to measure velocity. It can be used to monitor blood flow or other tissue movement in the body (cf. J. D. Briers, “Laser Doppler, speckle and related techniques for blood perfusion mapping and imaging”, Physiol. Meas. 22, R35 (2001)). By its very nature, the method normally measures the flow in the direction towards or away from the laser beam, e.g. in the axial direction of a catheter in devices known from the state of the art. The catheter device 100 presented here, however, renders it possible to measure a two- or three-dimensional flow with a single catheter, thus resolving all vector components of the blood flow velocity. Such a more comprehensive flow assessment enhances significantly the vascular interventionalist's or vascular interventional physician's ability to define the optimum treatment strategy.

Typical sizes of the catheter device 100 are such that it will be readily suitable for neurovascular applications: the fiber 1 (including core 2 and cladding) can be roughly 1 mm in diameter, and the distance from the fiber end through the two dichroic elements 11, 12 to the end of the device 100 will be of the order of 1 mm as well.

FIG. 2 shows a second embodiment of a catheter device 200, wherein the light source and the detector may be similar to those of FIG. 1 and are therefore not shown again. The light-splitting unit 201 of this embodiment comprises a grating 21 disposed at the outlet of the optical fiber 1, the grating having a suitably chosen blaze angle α. When a grating has a blaze angle, it is possible to concentrate most of the diffracted energy in a particular order for a given wavelength λ₁. For other wavelengths, the diffraction efficiency will be less and the light will be transmitted without changing direction. Changing the wavelength λ₁ thus changes the direction α of a partial light beam B1 that exits the splitting unit 201 together with a partial light beam B2 emitted in forward direction. The blood flow can therefore be probed in different directions. This is analogous to the situation with different wavelengths in FIG. 1. Two components of the blood flow vector can be resolved since there is only one blaze angle α. The angle α between the two partial beams B1 and B2 need not be 90′; provided there is a substantial difference, two components of the blood flow vector can be resolved.

FIG. 3 shows a third embodiment of a catheter device 300, in which a polarization-maintaining fiber 1 is used in combination with an optical polarizer 31 in a light-splitting unit 301. Such a (commercially available) fiber 1 can propagate two polarizations π₁, π₂ of light separately, with no cross-talk between these modes. Separation of the two polarizations can be achieved by an optical polarizer 31, such as polarizing beam splitter cubes or polarization-sensitive anisotropic gratings. The optical polarizer results in substantially different exit angles for two partial beams B1, B2 with two polarization directions (indicated by double arrows, of which one should be perpendicular to the drawing plane) of the primary light beam in the fiber 1. As a result, the two polarization directions of the light probe different directions in the blood flow. This is analogous to the situation with different wavelengths in FIG. 1. Two components of the blood flow vector can be resolved since there are two polarization directions for light in a polarization-maintaining fiber.

FIG. 4 shows a fourth embodiment of a catheter device 400 which combines the second and the third embodiment by arranging an optical polarizer 31 in series with a grating 21 in a light-splitting unit 401. A polarization-maintaining core 2 waveguides different colors around a wavelength λ₁, which colors are substantially close in wavelength (typically less than roughly a factor two). At the optical polarizer 31, one polarization direction π₁ is reflected into a first partial light beam B1 having a certain direction while the other polarization direction is transmitted as an intermediate second partial light beam B2. At the grating 21, one color λ₁ of the second partial light beam B2 is diffracted and leaves as a third partial light beam B3, while the residual light is transmitted as a fourth partial light beam B4.

Thus all three components of the blood flow vector can be resolved, without the need for three wavelength intervals or multiple dichroic elements. The direction of the partial light beams B1, B3 and B4 exiting the light splitting unit 401 is changed by changing either the polarization or the wavelength of the light. This reduces the volume and complexity of the optics at the end of the fiber.

In this embodiment, the optical polarizer 31 is ideally placed in front of the grating 21, as the light refracted from the grating will not propagate at a 90° angle with respect to the transmitted light. However, an embodiment with the optical polarizer after the grating with a blaze angle is also feasible.

The embodiments of the invention described above may be used in particular for dynamically assessing the blood flow near an aneurysm during an endovascular procedure. It should further be noted that the embodiments do not contain any metal parts and can therefore be used in an MR system.

Finally, it is pointed out that the term “comprising” in the present application does not exclude other elements or steps, that “a” or “an” does not exclude a plurality, and that a single processor or other unit may fulfill the functions of several means. The invention resides in each and every novel characteristic feature and each and every combination of characteristic features. Moreover, reference signs in the claims shall not be construed as limiting their scope.

1. A light-emitting apparatus (100, 200, 300, 400), comprising
a) an optical waveguide (1) for conducting a primary light beam (Bprim);
b) a light-splitting unit (101, 201, 301, 401) for splitting said primary light beam (Bprim) into at least two partial light beams (B1, B2, B3, B4) of different optical qualities that are emitted in different directions.

2. The light-emitting apparatus (100, 200, 300, 400) according to claim 1, characterized in that said optical quality comprises polarization and/or spectral composition.

3. The light-emitting apparatus (100, 200, 300, 400) according to claim 1, characterized in that the light-splitting unit (101, 201, 301, 401) comprises a splitting component selected from the group consisting of a dichroic beam splitter (11, 12), a grating (21), and an optical polarizer (31), for splitting an incident light beam (Bprim, B2) into a first and a second partial light beam (B1, B2, B3, B4) of different directions and different optical qualities.

4. The light-emitting apparatus (100, 200, 300, 400) according to claim 3, characterized in that the light-splitting unit (101, 201, 301, 401) comprises a further splitting component selected from the group consisting of a dichroic beam splitter (12), a grating (21), and an optical polarizer, for splitting the second partial light beam (B2) into a third and a fourth partial light beam (B3, B4) of different directions and different optical qualities.

5. The light-emitting apparatus (100, 200, 300, 400) according to claim 4, characterized in that the splitting components are dichroic beam splitters (11, 12) having the shape of prisms with a triangular base and oriented at a rotational angle of approximately 45° about the axis of the incident light beam (Bprim).

6. The light-emitting apparatus (100, 200, 300, 400) according to claim 3, characterized in that the splitting component is a grating (21) that has a blaze angle (α) for a particular wavelength (λ1).

7. The light-emitting apparatus (100, 200, 300, 400) according to claim 1, characterized in that it comprises a detector (4) for detecting a secondary light beam (Bsec) that comprises light collected by the light splitting unit (101, 201, 301, 401) from its surroundings.

8. The light-emitting apparatus (100, 200, 300, 400) according to claim 7, characterized in that the detector (4) is adapted for separately processing components of the secondary light beam (Bsec) of different optical qualities.

9. The light-emitting apparatus (100, 200, 300, 400) according to claim 8, characterized in that the detector (4) comprises an evaluation module (5) for determining the Doppler shift in at least one component of the secondary light beam (Bsec) with respect to a corresponding partial light beam (B1, B2, B3, B4).

10. The light-emitting apparatus (100, 200, 300, 400) according to claim 1, characterized in that it comprises a light source, particularly a laser light source (3), for emitting the primary light beam (Bprim) into the optical waveguide (1).

11. The light-emitting apparatus (100, 200, 300, 400) according to claim 10, characterized in that the laser light source (3) has a coherence length greater than 1 mm, preferably greater than 10 mm, most preferably greater than 100 mm.

12. Medical device comprising a light-emitting apparatus (100, 200, 300, 400) according to claim 1.

13. A method of measuring a flow velocity of a fluid, particularly of blood, comprising the steps of:
a) emitting at least two partial light beams (B1, B2, B3, B4) of different optical qualities in different directions from a measuring location into the fluid;
b) receiving a secondary light beam (Bsec) comprising components consisting of light from the partial light beams (B1, B2, B3, B4) reflected in the fluid;
c) determining a flow velocity of the fluid from a Doppler shift in said components of the secondary light beam (Bsec).
